(19) United States
(12) Patent Application Publication
(10) Pub. No.: US 2026/0053452 A1
(43) Pub. Date: Feb. 26, 2026

(54) X-RAY TUBE WITH REDUCED IMAGING EXPOSURE TIME

(71) Applicant: 3DIO, Inc., Orem, UT (US)

(72) Inventors: Douglas P. HANSEN, Spanish Fork, UT (US); Keith DECKER, Pleasant Grove, UT (US); Jacob CASSINAT, Provo, UT (US)

(21) Appl. No.: 18/812,352

(22) Filed: Aug. 22, 2024

(51) Int. Cl. A61B 6/40; A61B 6/51; H05G 1/20; H05G 1/30

(52) U.S. Cl. CPC A61B 6/405; A61B 6/51; H05G 1/20; H05G 1/30

(57) ABSTRACT

X-ray tubes that have a reduced exposure time for dental X-ray imaging and 3D dental imaging systems using these X-ray tubes are described. The X-ray tubes contain an anode, a filament, a cathode electrically connected to the filament, and a voltage source electrically connected to the cathode. The voltage source provides a high voltage to the anode relative to the cathode and filament control voltage. A low filament control voltage is used to generate electrons using a process of thermionic emission. These electrons are driven by a large electric field generated between the cathode and anode towards the target on the anode. A bias voltage is applied between the cathode and the filament to control the electron flow from the emitting filament to the anode. Such a configuration yields a switching speed ranging from about 1 ms to about 10 ms, allowing a short X-ray exposure time and a quicker overall imaging process for the 3D dental X-ray imaging systems. Other embodiments are described.

### FIELD

This application relates generally to X-ray equipment, including X-ray devices and X-ray tubes. More specifically, this application relates to X-ray tubes that have a reduced exposure time for dental X-ray imaging and 3D dental imaging systems using these X-ray tubes.

### BACKGROUND

X-ray imaging systems typically contain an X-ray source and an X-ray detector. X-rays (or other types of radiation used for imaging) are emitted from an X-ray tube in the X-ray source and impinge on the X-ray detector to provide an X-ray image of the object or objects that are placed between the X-ray source and the detector. The X-ray detector is often an image intensifier or even a flat panel digital detector.

Intra-oral radiography is a standard imaging technique in dentistry, with bite-wing and periapical X-rays considered the standard of care in dental practice. However, there are many features of the tooth anatomy that are not visible in standard intra-oral radiographs since they are only two-dimensional (2D) projections of a three-dimensional (3D) structure. Accordingly, 3D imaging is often used in some dental procedures. One form of 3D imaging, cone-beam computed tomography (CBCT), is becoming widely used in dental imaging. In CBCT, a patient's head is positioned between a large imaging detector and an opposing X-ray source. The detector and source rotate around the head while taking multiple 2D images. Using these 2D images, a 3D image of the patient's oral and maxillofacial anatomy can be reconstructed. This technique works very well for imaging the entire oral cavity and displaying the spatial relationships between the teeth and other bony structures located in the head of a patient. But it is often not used to image just a few (or a single tooth) because of the increased radiation that the patient is subjected to.

### SUMMARY

This application relates generally to X-ray tubes that can be used in X-ray devices. More specifically, this application relates to X-ray tubes that have a reduced exposure time for dental X-ray imaging and 3D dental imaging systems using these X-ray tubes. The X-ray tubes contain an anode, a filament, a cathode electrically connected to the filament, and a voltage source electrically connected to the cathode. The voltage source provides a high voltage to the anode relative to the cathode and filament control voltage. A low filament control voltage is used to generate electrons using a process of thermionic emission. These electrons are driven by a large electric field generated between the cathode and anode towards the target on the anode. A bias voltage is applied between the cathode and the filament to control the electron flow from the emitting filament to the anode. This bias voltage between the cathode and the filament is lower than the voltage between the cathode and the anode, allowing for faster control of X-ray emission and a shorter overall exposure time, resulting in a quicker imaging process for the 3D X-ray imaging system. Such a configuration yields a switching speed ranging from about 1 ms to about 10 ms, allowing a short X-ray exposure time and a quicker overall imaging process for the 3D dental X-ray imaging systems.

### BRIEF DESCRIPTION OF THE DRAWINGS

The following description can be better understood in light of the Figures which show various embodiments and configurations of the X-ray tubes and X-ray devices in which they are used.

FIG. 1 shows a view of some embodiments of a 3D X-ray imaging system;

FIG. 2 shows a view of some embodiments of an X-ray head of a 3D X-ray imaging system;

FIG. 3 shows a view of other embodiments of an X-ray head of a 3D X-ray imaging system;

FIG. 4 shows other embodiments of the housing and components contained in the housing of an X-ray head of a 3D X-ray imaging system.

FIG. 5 shows some embodiments of a 3D X-ray imaging system mounted to equipment in a dental office; and

FIG. 6 shows some embodiments of the X-ray tubes used in the 3D X-ray imaging systems.

Together with the following description, the Figures demonstrate and explain the principles of the structures and methods described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices.

### DETAILED DESCRIPTION

The following description supplies specific details in order to provide a thorough understanding. Nevertheless, the skilled artisan will understand that the described X-ray systems can be implemented and used without employing these specific details. Indeed, the described systems and methods can be placed into practice by modifying the described systems and methods and can be used in conjunction with any other apparatus and techniques conventionally used in the industry. For example, while the description below focuses on X-ray tubes that can be used in imaging systems for dental imaging, they can be used for other purposes such as medical imaging, veterinary imaging, industrial inspection applications, and anywhere where X-ray radiography equipment is currently being used to generate a standard 2D X-ray image.

In addition, as the terms on, disposed on, attached to, connected to, or coupled to, etc. are used herein, one object (e.g., a material, element, structure, member, etc.) can be on, disposed on, attached to, connected to, or coupled to another object—regardless of whether the one object is directly on, attached, connected, or coupled to the other object or whether there are one or more intervening objects between the one object and the other object. Also, directions (e.g., on top of, below, above, top, bottom, side, up, down, under, over, upper, lower, lateral, orbital, horizontal, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

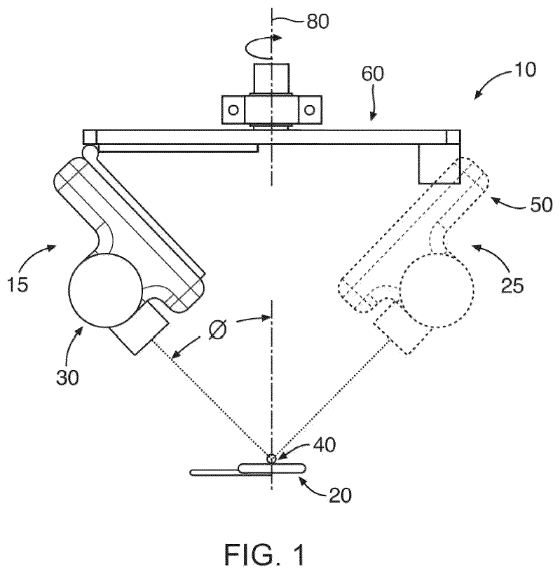

Some embodiments of the 3D dental X-ray imaging system containing X-ray tubes that have a reduced exposure time are illustrated in FIG. 1. FIG. 1 shows the geometry of a 3D dental imaging system with a rotation scheme shown with the X-ray source at position 15 and position 25 around axis of rotation 80. In FIG. 1, the 3D dental imaging system 10 comprises an imaging detector 20 that is located inside the mouth (not shown). The imaging detector 20 can be substantially stationary adjacent to the tooth (or teeth) 40 of a patient, or even completely stationary relative to the tooth using any stabilizing mechanism such as a bite block or other sensor holder device. The 3D imaging system 10 also contains an X-ray source 30 that is optionally located within a housing 50. The housing 50 can be connected to a support arm 60.

The 3D dental imaging system 10 can contain any X-ray source 30 and X-ray detector 20 that allows the system 10 to take multiple 2D X-ray images or radiographs. The X-ray source 30 can contain any source that generates and emits X-rays, including a standard stationary anode X-ray source, micro-focus X-ray source, rotating anode X-ray source, and/or a carbon nanotube or micro-machined (Spindt cathode) X-ray source. In some embodiments, the X-ray source can operate with about 40 kV to about 90 kV and from about 1 mA to about 10 mA. In other embodiments, the X-ray source can operate with about 55 kV to about 75 kV and between about 3 mA and about 9 mA. In still other embodiments, the X-ray source can operate with about 60 kV to about 70 kV and between about 4 mA and about 7 mA. In some embodiments, the X-ray source and X-ray detector can be made modular so that different sizes and types of X-ray sources and X-ray detectors can be used.

The X-ray detector 20 can contain any detector that detects X-rays, including an image intensifier, CCD array, CMOS/scintillator array, and/or a digital flat panel detector. In some configurations, the detector can have a substantially square shape with a length on one side ranging from about 2.5 cm to about 6 cm. In other configurations, though, the X-ray detector 20 does not need to have a substantially square shape but can have a rectangular shape to roughly match the size of a tooth of a patient. In yet other embodiments, though, the X-ray detector 20 does not need to have a substantially square or rectangular shape.

In some configurations, the X-ray detector 20 can be synchronized and/or aligned with the X-ray source 30 so that the X-ray system 10 can take multiple images with high image efficiency. This synchronization can be performed by controlling both the X-ray detector and the X-ray source using an internal or external controller, such as a computer, or by configuring the detector to collect data when it first detects X-rays, thereby controlling the timing of the X-ray source needs so it emits the X-ray pulses when desired.

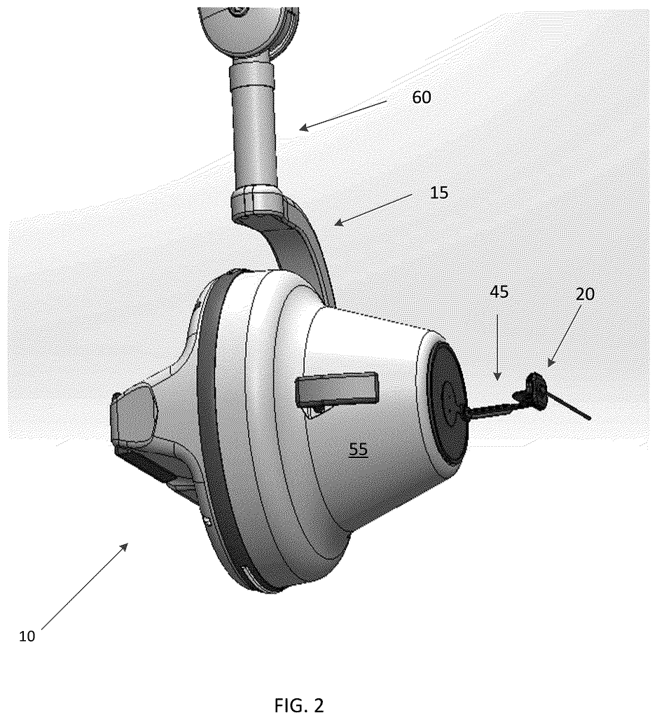

Other embodiments of the 3D dental X-ray system are shown in FIG. 2. The 3D dental X-ray imaging system 10 can contain an X-ray head 55 that contains an X-ray source 30 (not shown) and high voltage electronics (not shown). The high voltage electronics can provide typically between about 40 KV volts and about 200 KV for most medical applications, and/or about 50 KV up to about 70 KV for most dental applications, whether DC or AC current. The X-ray imaging systems also contain an arm 60 that is coupled to the X-ray head 55. The X-ray head 55 can be configured to be removably connected to the arm 60 using a yoke 15 so that when the X-ray head 55 is disconnected, it can be removed from the remainder of the X-ray system 10.

The X-ray head 55 can also be attached to an X-ray detector 20. The X-ray detector 20 can contain any detector (or sensor) that detects X-rays, including an image intensifier, CMOS camera, and/or a digital flat panel detector. The X-ray detector 20 can be connected to the X-ray head 55 using an aligner 45 that helps keep the X-ray detector 20 properly positioned with respect to the tooth of a patient while a dental X-ray image is taken, or that serves similar functions in medical X-ray applications.

In some embodiments, the X-ray source and X-ray detector can be made modular so that different sizes and types of X-ray sources and X-ray detectors can be used. This allows the X-ray source 30 and the X-ray detector 20 to be easily replaceable in the X-ray system 10.

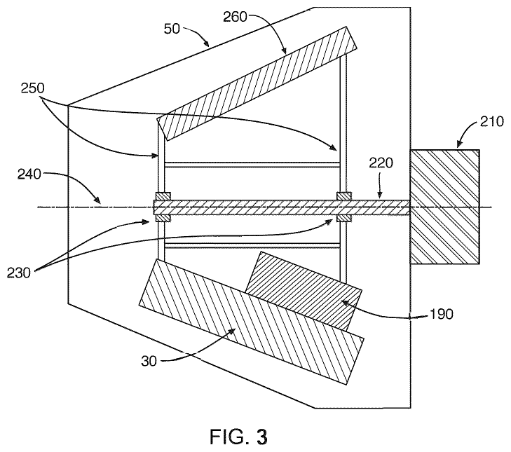

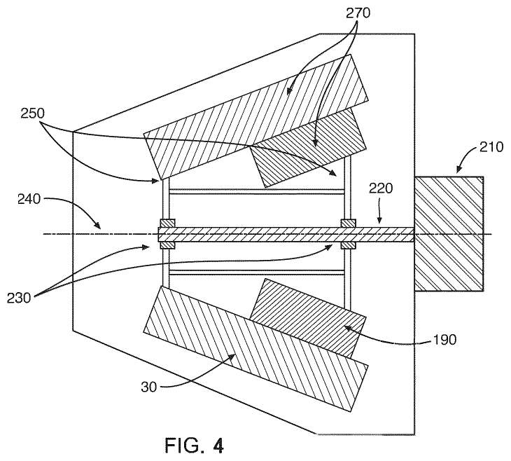

Other embodiments of the X-ray head are shown in FIGS. 3-4. As shown in detail in FIG. 3, the X-ray source 30 can be contained in housing 50. The housing 50 can be configured with a first part enclosing the X-ray source 30 as shown in FIG. 3. The housing 50 also encloses a second part that contains a counterweight 260 for the X-ray source 30, power electronics 190, and other components, which facilitates smooth vibration-free rotary motion of the source 30. The X-ray source 30 and its associated power electronics 190 and the counterweight 260 are located as necessary on rotating mechanical assembly 250 which supports the X-ray source 30, the power electronics 190, counterweight 260, and other components (not shown) to properly balance the rotating mechanical assembly 250. The rotating mechanical assembly 250 is mounted to axle 220 (or other mechanical device to support the mechanical assembly) with an axis of rotation 240 using the bearings and/or electric motor assembly 230 to enable drive rotation of the mechanical assembly 250.

As shown in FIG. 3, the housing 50 can be configured so that it is a single part that encloses both the X-ray source 30 and these components. In other configurations, the housing can be separated into different parts to contain the X-ray source 30 and other components. As shown in FIG. 3, the electronic components for control and power conditioning 210 can be located just outside of the housing 50. In other embodiments, these electronic components 210 can be located on the support arm or other convenient location. In yet other embodiments, these electronic components 210 can be located internal to the housing 50.

In some embodiments, multiple X-ray sources can be used in the 3D imaging systems. In these embodiments, as shown in FIG. 4, multiple X-ray sources (30, 270) would enable a reduction in the mechanical rotation speed required to cover all of the desired source positions needed to generate the 3D image. These sources could be fired in an alternating manner or otherwise as required to obtain all of the desired 2D images from the various X-ray source locations within the head. The remainder of the components in FIG. 10 can be similar to those shown in FIG. 4, with the exception that the second X-ray source and its associated high voltage electronics 270 have replaced the counterweight 260.

The use of multiple X-ray sources 30 within the housing 50 also provides the benefit of reduced motion blur in the X-ray images obtained since the X-ray source is moving at a lower velocity than would be required with a single source. The use of multiple, substantially identical sources 30 would also negate the requirement for a counterweight since the multiple sources can be positioned to result in a balanced rotational system. More than two X-ray sources could be incorporated into the 3D imaging system, with the full 360 degrees of the circle being divided by the number of sources used so that the multiple sources are distributed evenly around the circular frame on which they are mounted. Of course, the use of multiple sources will increase the system overall cost and complexity, so the needs and constraints of the intended use will need to be considered in choosing the number of sources to be included within a particular 3D imaging system.

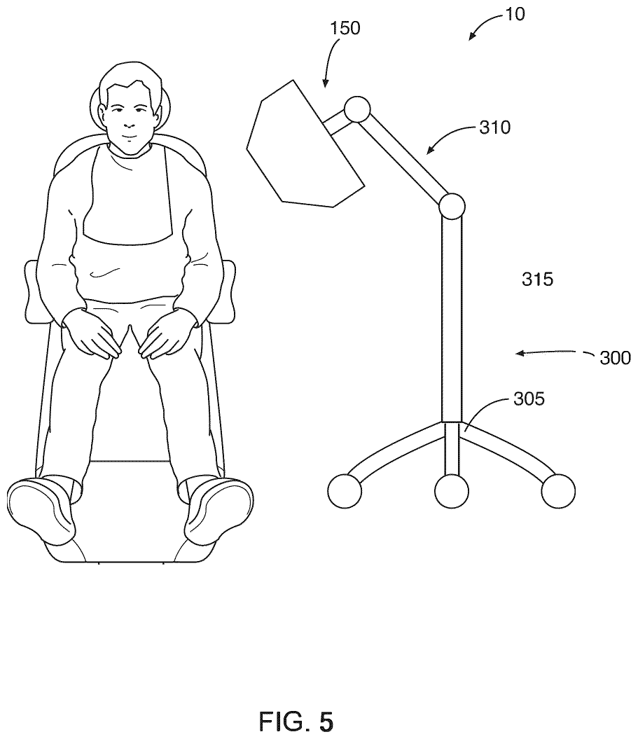

In some configurations, the 3D dental imaging systems can contain a removable power source (such as a battery) and optionally a power supply. In these configurations, the power source and the power supply can be located on or in any supporting structure which the 3D imaging systems might be used with. For example, the supporting electronics for the power source and the power supply, as well as the supporting electronics for the image display and for the wireless data upload described herein, can also be located internal or external to a support structure to which the housing 50 is connected, such as stand 300 shown in FIG. 5. Thus, in these configurations, the system 10 does not require an external power cord. Incorporating the power source (i.e., the battery), the power supply, and the supporting electronics all in or on the external structure allows the 3D imaging systems to be portable and moved from one dental station to another. With such a configuration, the power source can easily be replaced or swapped. Of course, if needed, the 3D imaging system 10 can be configured so that it is alternately, or additionally, powered using external power from a power cord that is plugged into a wall outlet. In other configurations, multiple power supplies can be provided for the source, detector, and control electronics.

The support arm 60 can have any configuration that allows the X-ray source 30 in the housing to direct X-ray beams at the desired angle through the tooth (or teeth) and on the detector 20. In the embodiments shown in FIG. 1, the support arm 60 has a substantially straight configuration with the housing 50 connected to an end thereof. In other configurations, the support arm need not be straight and can have jointed or articulated sections such as those shown in FIG. 2. In yet other configurations, the housing 50 can be connected to the support arm 60 at any location other than its end.

In some embodiments the 3D dental imaging systems with the X-ray tubes can be attached to an external support structure, as illustrated in FIG. 5. In this Figure, the 3D imaging system 10 with a frame 150 can be connected to a stand 300. The stand 300 contains a base 305 and an arm 315 extending upwards towards an extension 310. The extension 310 is connected to the joint which is, in turn, connected to the frame 150 of the 3D imaging system 10. In other configurations, the 3D imaging system 10 can be connected to a movable support structure. In such configurations, the movable support structure can be configured to move across a floor while supporting the 3D imaging system 10. Thus, the movable support structure can comprise one or more wheels, shelves, handles, monitors, computers, stabilizing members, limbs, legs, struts, cables, and/or weights (to prevent the weight of the imaging arm and/or any other component from tipping the movable support structure). Thus, the movable support structure could comprise a wheeled structure connected to a stand that contains the joint that is connected to the frame 150 of the 3D imaging system 10.

The volume and weight of the 3D X-ray imaging system should be minimized as much as possible for ease of use and ease of alignment. To reduce the size and/or weight, the 3D X-ray imaging system can be equipped with small and lightweight components. Over the last decade, there have been significant innovations in miniaturization of X-ray tubes. These light weight sources can greatly simplify the task of motion automation for the 3D X-ray imaging systems described herein. In addition, newer CMOS detectors are much more sensitive, resulting in less dose to the patient than required with conventional CCD designs. The new CMOS detectors also have very high read-out speeds allowing for rapid collection and transmission of multiple 2D images. As well, CMOS detectors with capability of at least 5 (or more) frames per second can be used.

To achieve the small and lightweight 3D X-ray imaging systems described herein, the X-ray source (or X-ray tube) can fit within a volume of about 13 cm×about 7 cm×about 8 cm and weigh less than about 1.9 Kg in some configurations. One way to achieve X-ray sources that meet these requirements can be to use a carbon-nanotube or Spindt-cathode (micro-machined silicon or similar technology) electron source within the X-ray source as this technology will simplify the X-ray tube design, enabling a smaller X-ray source and faster X-ray pulses.

In some embodiments, the X-ray source 30 can contain an X-ray tube that is capable of reducing the exposure time of the X-ray system 10. Some 3D X-ray imaging systems utilize X-ray generators that emit high levels of instantaneous, pulsed radiation to produce numerous (often more than 100) individual 2D projection images of a patient, thereby achieving a reasonable radiation dose for a reconstructed 3D image and a reasonable data acquisition time. The electronic circuits used are designed with short exposure times (and short pulse times) and the X-ray tubes device can be configured for gridded control. With gridded control X-ray tubes, a third electrode can be used in addition to the filament that generates the electrons and the anode. This third electrode (the gate) controls the flow of electrons from the filament to the target.

As described herein, the 3D X-ray imaging systems take multiple 2D images to be used in reconstructing a 3D image. In some configurations, the X-ray systems can take anywhere from about 15, about 20, about 25, about 30, about 35, about 40, about 50, or even about 60 2D images in order to render a 3D image. In other configurations, the number of 2D images can be any combination or sub-combination of these numbers. There are even some configurations (known as Portray) that only need to take 7 images using a carbon nanotube X-ray source.

Since the 2D images are taken of a dental patient who is not constrained, the 2D images need to be taken as quickly as possible, including in terms of overall time, the time to acquire the series of images, as well as the acquisition of each individual image. Limiting the overall acquisition time, as well as the duration of each individual image acquisition, can be helpful to avoid patient movement and/or patient discomfort. Thus, the 3D X-ray imaging systems should take the 2D images in the range of about 7 to about 22 frames/second and with an X-ray pulse time ranging from about 8 ms to about 35 ms, which is much lower than the 100 ms or more often used by some conventional dental X-ray systems. Under these conditions, the X-ray tubes can have a short rise and fall time so that the majority (at least about 75% of the pulse duration and higher) of each individual X-ray pulse is at the proper intensity to obtain a good, consistent exposure of the sensor and a good resulting 2D image. Accordingly, for an X-ray pulse of about 35 ms, the rise and fall times should be less than about 6 ms, less than about 5 ms, or even less than about 4 ms. Similarly, for an X-ray pulse duration of about 8 ms, the rise and fall times should be less than about 1 ms, less than about 0.67 ms, or even less than about 0.5 ms. For X-ray pulse times between 8 ms and 35 ms, the rise and fall times can be adjusted to be between the amounts listed above.

These quick X-ray pulse times also reduce motion blur that would otherwise be incurred in the 2D images if the individual X-ray pulses were longer. This short, high-intensity pulse capability is therefore helpful in dental tomosynthesis where the number of 2D images acquired must be limited for reasons of reduced patient radiation dose, reasonable data acquisition times, and also reasonable 3D reconstruction times. The reduced motion blur from short X-ray pulses also helps achieve diagnostic quality 3D reconstruction of the teeth that are being imaged.

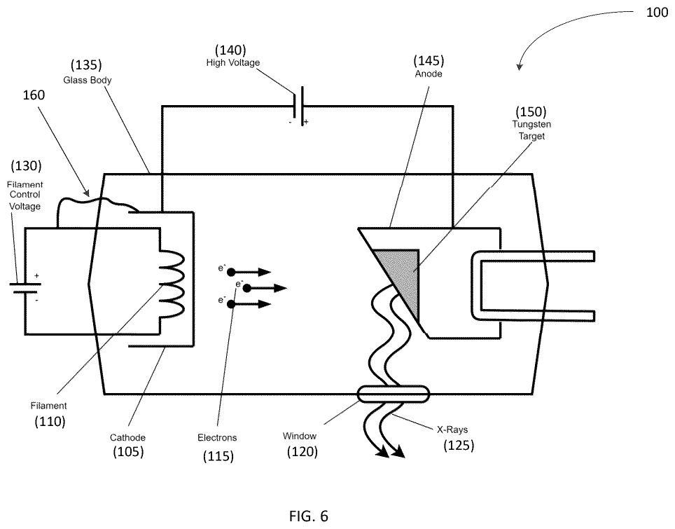

Generally, the X-ray tubes used in the 3D X-ray imaging systems described herein contain an anode, a filament, a cathode electrically connected to the filament, and a voltage source electrically connected to the cathode. The X-ray tubes can use a gridded tube configuration. The X-ray tubes electrically connect the filament to the cathode to simplify the electrical controls while achieving the desired performance benefit. An example of these X-ray tubes is shown in FIG. 6 as X-ray tubes 100. The voltage source provides a high voltage to the anode relative to the cathode and filament control voltage. A low filament control voltage can be used to generate electrons using a process of thermionic emission. These electrons can be driven by a large electric field generated between the cathode and anode towards the target on the anode. A bias voltage can be applied between the cathode and the filament to control the electron flow from the emitting filament to the anode. This bias voltage between the cathode and the filament is lower than the voltage between the cathode and the anode, allowing for faster control of X-ray emission and a shorter overall exposure time, resulting in a quicker imaging process for the 3D X-ray imaging system.

These X-ray tubes 100 can achieve these results by electrically floating one end of the X-ray tube cathode and connecting the other end to the filament. As shown in FIG. 6, the X-ray tubes 100 contain a body 135 that encloses the cathode 105 and the anode 135. In the illustrated embodiments, the body 135 is made of glass or any similar material. Electrons are emitted from the cathode 105 and impinge on the target 150 of the anode 145, thereby generating X-rays 125 that are emitted from the X-ray tube 100 through a window 125 in the body 135. In the illustrated embodiments, the target 150 can be made of tungsten (W) or another material such as copper (Cu), molybdenum (Mo), or tantalum (Ta).

The X-ray tube 100 contains a cathode 105 that is located near the filament 110. The cathode 105 can be separated from the filament by any distance that can generate about 1 kV to about 5 kV potential between them. In some embodiments, the cathode 105 and the filament 120 are separated by any distance that yields a potential of about 1.25 kV, about 1 kV, or even about 0.75 kV. The cathode 105 is also electrically connected to the filament 110 by using connecting wire 160.

As shown in FIG. 6, a high positive voltage is applied to the anode. At the same time a filament control voltage 130 is applied to generate electrons from the filament. By biasing the cathode appropriately, the electron flow can be controlled with a high level of precision. The connection wire 160 allows the system to maintain the same voltage reference across both voltage potentials [e.g., high voltage (140) and filament control voltage (130)]. This connection wire (160) allows for the applied cathode voltage to act as a gate. By applying a voltage potential to the cathode relative to the filament, the flow of electrons can be allowed or stopped regardless of the applied voltage to the anode. X-rays 125 can be generated when the negative potential applied to the floating cathode 105 creates a circuit that applies the variable kilovolt (kV) potential described herein. That variable potential ranges from a low voltage when no X-rays are generated, to the 1 kV potential that generates the X-rays to emit from the cathode.

Using these X-ray tubes 100, the switching speeds can be reduced relative to some conventional X-ray tubes. Rather than having to increase the voltage on the high voltage (140) circuit from 0 to the target voltage, the voltage applied to the cathode only needs to be decreased relative to the filament control voltage 130 to the emitting voltage. In some embodiments, the switching speeds can be reduced to less than about 1 ms for each X-ray image acquired. In other embodiments, the switching speeds can be reduced to less than about 10 ms for each X-ray image acquired when the X-rays 125 need to be emitted from the tube. These switching speeds are unavailable to some conventional stationary tube configurations because the high voltage circuit is maintained at a 0 voltage rather than the target kV voltage used in the X-ray tubes 100.

Using the gated X-ray tubes 100 also allows the selection of a smaller focal spot, meaning the spot where the electrons are focused on the anode to produce X-rays. Since electrons accelerated with the high voltage and then colliding with the anode creates the X-rays, the size of the focal spot is equivalent to the size of the X-ray source. It is known in geometric optics that the size of the source is a factor in determining the resolution of an optical system.

Some conventional extra-oral dental tomosynthesis diagnostic systems, such as CBCT systems, contain X-ray tubes that utilize a 0.8 mm focal spot. A focal spot of this size is one factor leading to lower resolution of the images typically ranging from about 100 microns to about 150 microns. The gated X-ray tubes 100, however, enable the use of smaller focal spots, such as about 0.6 mm, about 0.4 mm, or even about 0.2 mm to about 0.3 mm. In some embodiments, the focal spot can be any range, combination, or sub-combination of these numbers. This lower focal spot size can be obtained because the gated X-ray tubes provide for additional control over the electrons, allowing them to be brought into a tighter focus. Using a smaller focal spot allows for a higher image resolution, but can increase the costs since the system resolution will also depend on other factors, including sensor performance (i.e. pixel size), the distances between the X-ray source, the object, and the X-ray sensor (which can determine the system magnification and spectral performance of the X-ray source). But if these other factors are controlled and kept constant, reducing the focal spot by a factor of about two can increase the optical resolution by a factor of about two which will produce a higher-resolution image. Accordingly, in some configurations of the X-ray systems using the gated X-ray tubes 100, the X-ray systems are able to obtain an image resolution of about 35 microns to about 55 microns.

During the reconstruction process, the multiple 2D images are effectively “averaged” such that random noise in the images can be significantly reduced. This averaging effect can also be utilized to reduce the radiation dose. For example, in some embodiments the sensor gain for the X-ray detector in the X-ray systems can be increased. Increasing the gain in an electronic device, such as an amplifier or a sensor, will increase random noise in the signal. However, this random noise can be eliminated through averaging the signal. Thus, increasing the sensor gain can also increase the random noise in each individual 2D image, but that noise can also be averaged out during the reconstruction process so that the resulting 3D image is clear and precise with low noise. In this way, the patient radiation dose can be significantly reduced compared to what would be received by the patient if each 2D image was acquired at a similar dose as some conventional X-ray systems. In some configurations, if the sensor gain is increased by a factor of 10 over that which is used in some conventional intra-oral dental sensors, the radiation dose required to provide a proper exposure for each 2D image would be reduced by about 1/10^{th} (though this image would exhibit more random noise). In these configurations, if the 3D imaging system were to acquire 40 2D images with 10× higher gain setting, the net radiation dose delivered to the sensor (and the patient) would be only about 4× that of a corresponding conventional image rather than 40×, which is a significant reduction in dose. Other adjustments to the exposure parameters of the sensor can also be made to optimize the reconstructed image quality versus the patient dose, leading to the ability to produce a high resolution reconstructed 3D image while delivering a radiation dose to the patient that will be between about 1.7× to about 4× less than that of a corresponding conventional 2D image. Other increases to the sensor gain are also possible, so the gain might be increased by a factor of about 2.5 times, about 5 times, about 7.5 times, about 10 times, about 12.5 times, about 15 times, or even about 20 times over that of some conventional 2D sensors.

The X-ray tubes 100 can sometimes be referred to as gated X-ray tubes (or gated tubes). Gated tubes are typically not used in dental X-ray systems because additional circuitry is needed for controlling the cathode and filament and no significant performance benefit would be obtained from this expense. Single pulse X-ray dental systems are typically driven using a single step-up transformer, a voltage multiplier circuit, and a gate control circuit. Accordingly, there needs to be a gate control multiplier to adjust the voltage of the gate relative to the voltage of the cathode/filament. As well, there needs to be a low voltage input control circuit to make sure that the gate control circuit operates when the high voltage is applied to the X-ray tube, otherwise the tube would not operate. But these problems are outweighed in the dental X-ray systems described herein by the shorter exposure times, fast rise times and fall times, high instantaneous power, and higher image resolution that can be achieved, as well as the rapid acquisition of 2D X-ray projection data and reduced patient exposure to radiation.

The X-ray tubes 100 and the 3D dental X-ray imaging systems in which they are used exhibit several helpful features. One helpful feature is the shorter switching times allowed with the X-ray tubes 100. These shorter switching times, in turn, result in a short total acquisition time for the X-ray images for any given number of images. Thus, the time needed from the start of X-ray pulse to the time the X-rays reach the target intensity can be minimized. Indeed, the configurations described herein allow for rise times from about 1 ms to about 10 ms that would be challenging for conventional dental X-ray tubes to obtain. In other configurations, this rise time can range from about 1 ms to about 7.5 ms, about 1 to about 5 ms, or even about 1 ms to about 3 ms. In still other configurations, this rise time can be any combination or sub-combination of these numbers.

As well, these shorter switching times result in reduced motion blur because the X-ray images can be taken in a shorter amount of time. Generally, the longer the detector is exposed to X-rays while in motion, the more an X-ray image is blurred. So a shorter switching time leads to shorter exposure to X-rays and, therefore, sharper images. These shorter switching times also result in a reduced patient dose because less X-rays are emitted from the anode during the switching time.

Another helpful feature is the improvement over the limitations of image artifacts and low resolution found in dental extra-oral tomosynthesis and CBCT procedures. Using the X-ray tubes 100 will reduce exposure times and increase frame rate to match a more efficient, high frame rate digital sensor. The X-ray tube 100 can be pulsed to synchronize with the X-ray detector so that the detector is ready to receive an X-ray exposure when the X-rays are emitted from the anode. The 3D imaging systems described herein can be configured so that the X-ray source can move a predetermined amount around an arc in the X-ray head between each X-ray image capture. A registration mechanism can be used around the arc to trigger the X-ray pulses at the proper locations around the arc. The movement of the X-ray source can be configured so that there is sufficient time for the X-ray detector to process the image data from a first X-ray exposure and then prepare to receive X-rays again before a second X-ray exposure occurs. At the same time, the X-ray power from the X-ray source has to be limited because the weight of the X-ray head has to be as light as possible. These factors create a situation where the X-ray pulse should be reasonably short, but not so short that the detector can't capture a sufficient image. In some configurations, the X-ray pulse can range from about 5 ms up to 500 ms while in other configurations the X-ray pulse can range from about 10 ms up to 40 ms. As well, the X-ray pulse must provide sufficient X-ray flux to provide a good exposure for the detector during each pulse. To render a sufficient 3D image, at least 5 pulses are needed in a short time frame because of patient movement. This short time frame can be under about 5 seconds in some configurations and less than about 3 seconds in other configurations.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. An X-ray tube for a 3D dental imaging system, comprising:
an anode:
a filament;
a cathode electrically connected to filament; and
a voltage source electrically connected to the cathode;
wherein the X-ray tube has a switching speed ranging from about 1 ms to about 10 ms for each X-ray image acquired.

2. The X-ray tube of claim 1, wherein the switching speed ranges from about 1 ms to about 7.5 ms.

3. The X-ray tube of claim 1, wherein the switching speed ranges from about 1 ms to about 5 ms.

4. The X-ray tube of claim 1, wherein the switching speed ranges from about 1 ms to about 3 ms.

5. The X-ray tube of claim 1, wherein the voltage source provides a high voltage to the anode and a low voltage to the filament sufficient to maintain the filament with a filament control voltage ranging from about 5 V to 50 V.

6. The X-ray tube of claim 1, wherein the voltage difference between the filament and the cathode ranges from about 0.75 kV to about 5 kV.

7. The X-ray tube of claim 1, wherein the voltage difference between the filament and the cathode ranges from about 1 kV to about 1.25 kV.

8. The X-ray tube of claim 1, wherein a negative potential is applied to the cathode to create a circuit that applies a variable kV potential to the X-ray tube.

9. The X-ray tube of claim 1, wherein the X-ray tube can emit at least 5 X-ray pulses in a time ranging from about 3 seconds to about 5 seconds.

10. The X-ray tube of claim 1, wherein the focal spot of the X-ray tube ranges from about 0.2 mm to about 0.6 mm.

11. A 3D dental imaging system, comprising:
an X-ray detector; and
an X-ray source aligned with the X-ray detector, the X-ray source containing an X-ray tube comprising:
an anode:
a filament;
a cathode electrically connected to filament; and
a voltage source electrically connected to the cathode;
wherein the X-ray tube has a switching speed ranging from about 1 ms to about 10 ms for each X-ray image acquired.

12. The imaging system of claim 11, wherein the switching speed ranges from about 1 ms to about 7.5 ms.

13. The imaging system claim 11, wherein the switching speed ranges from about 1 ms to about 5 ms.

14. The imaging system claim 11, wherein the switching speed ranges from about 1 ms to about 3 ms.

15. The imaging system of claim 11, wherein the voltage source provides a high voltage to the anode and a low voltage to the filament sufficient to maintain the filament with a filament control voltage ranging from about 5 V to 50 V.

16. The imaging system of claim 11, wherein the voltage difference between the filament and the cathode ranges from about 0.75 kV to about 5 kV.

17. The imaging system of claim 11, wherein the voltage difference between the filament and the cathode ranges from about 1 kV to about 1.25 kV.

18. The imaging system of claim 11, wherein a negative potential is applied to the cathode to create a circuit that applies a variable kV potential to the X-ray tube.

19. The imaging system of claim 11, wherein the X-ray tube can emit at least 5 X-ray pulses in a time ranging from about 3 seconds to about 5 seconds.

20. The imaging system of claim 11, wherein the focal spot of the X-ray tube ranges from about 0.2 mm to about 0.6 mm.
